(19) United States
(12) Patent Application Publication
(10) Pub. No.: US 2018/0140311 A1
(43) Pub. Date: May 24, 2018

(54) Targeting locations in the body by generating echogenic disturbances

(71) Applicant: The Medical Research, Infrastructure and Health Services Fund of the Tel Aviv Medical Center, Tel Aviv (IL)

(72) Inventors: Ronny Winshtein, Ramat-Ha'Sharon (IL); Yochai Edlitz, Yavne (IL); Roy Gigi, Tel Aviv (IL)

(21) Appl. No.: 15/573,495

(22) PCT Filed: May 19, 2016
(86) PCT No.: PCT/IB2016/052934

§ 371 Date: Nov. 13, 2017

Related U.S. Application Data
(60) Provisional application No. 62/165,694, filed on May 22, 2015.

(51) Int. Cl. A61B 17/17

(52) U.S. Cl. CPC A61B 17/1725; A61B 17/1707; A61B 17/744; A61B 17/1703; A61B 17/1796

(57) ABSTRACT

Surgical apparatus includes a transducer (50) configured to be inserted into a cavity (28) inside a bone (22) within a body of a living subject and to engage an inner wall of the cavity at a selected location within the cavity. A drive circuit (38) is coupled to apply a drive signal to the transducer so as to cause an echogenic movement of the bone at the selected location.

### CROSS-REFERENCE TO RELATED APPLICATION

This application claims the benefit of U.S. Provisional Patent Application 62/165,694, filed May 22, 2015, which is incorporated herein by reference.

### FIELD OF THE INVENTION

The present invention relates generally to invasive medical devices, systems and methods, and particularly to techniques for identifying, marking and/or reaching a target location within the body of a living subject.

### BACKGROUND

Since the development of intramedullary nails for use in orthopedic surgery to manage bone fractures, it has been common practice to fix the nail to the bone by inserting locking screws through holes drilled through the bone in alignment with fixation holes provided transversely through the nail. This procedure has presented technical difficulties, as the distal fixation holes and their spatial direction are difficult to localize and to align with surgical drills and placement instruments, using current imaging means. The common solution to this problem is to drill the holes in the bone under X-ray or fluoroscopic guidance, often in combination with complex mechanical alignment devices such C-arms and stereotactic frames. This approach still suffers from imprecise alignment and increased radiation exposure of the surgeon, other operating room personnel, and the patient.

In response to these difficulties, a number of alternative approaches have been developed to guide the surgeon in finding the correct location and direction for drilling the bone in alignment with the fixation holes at the distal end of an intramedullary nail (referred to in the art as “distal targeting”). For example, U.S. Pat. No. 7,060,075 describes a distal targeting system in which a hand-held location pad is integral with a guide section for a drill or similar surgical instrument, and has a plurality of magnetic field generators. A sensor, such as a wireless sensor, having a plurality of field transponders, is disposed in an orthopedic appliance, such as an intramedullary nail. The sensor is capable of detecting and discriminating the strength and direction of the different fields generated by the field generators. Control circuitry, preferably located in the location pad, is responsive to a signal of the sensor, and determines the displacement and relative directions of an axis of the guide section and a bore in the orthopedic appliance. A screen display and optional speaker in the location pad provide an operator-perceptible indication that enables the operator to adjust the position of the guide section so as to align its position and direction with the bore.

Other distal targeting techniques use ultrasonic sensing. For example, U.S. Pat. No. 5,957,847 describes an apparatus for detecting a lateral locking hole of an intramedullary nail that includes a targeting device. The targeting device has a support lever having a slider attached thereto. An ultrasonic probe is mounted at the lower end of the support lever. An ultrasonic wave is transmitted and received by a transceiver of the ultrasonic probe while moving the slider in a direction perpendicular to the axis of the intramedullary nail by a screw. The position of the lateral locking hole of the intramedullary nail is detected by the height of the echo of the ultrasonic wave.

As another example. PCT International Publication WO 2010/116359 describes a device for orienting a bone cutting tool with respect to a locking screw hole of an intramedullary nail inserted within a bone. The device includes a device body with a cutting path for a cutting device and a distal end portion adapted for positioning against a surface of the bone. The device also includes an ultrasound probe holder, which serves for aligning the cutting path with the screw locking feature using at least one ultrasound signal of at least one ultrasound probe attached to the device.

### SUMMARY

Embodiments of the present invention that are described hereinbelow provide improved methods for identifying a location within a living body, such as a fixation hole in an intramedullary nail, as well as devices and systems for use in such identification.

There is therefore provided, in accordance with an embodiment of the invention, surgical apparatus, including a transducer configured to be inserted into a cavity inside a bone within a body of a living subject and to engage an inner wall of the cavity at a selected location within the cavity. A drive circuit is coupled to apply a drive signal to the transducer so as to cause an echogenic movement of the bone at the selected location.

In a disclosed embodiment, the transducer includes a piezoelectric crystal. Alternatively, the transducer includes a mechanical vibrator. Further alternatively, the transducer is configured to apply pulses of thermal energy to the inner wall.

In one embodiment, the transducer is further configured to thin the bone at the selected location.

In some embodiments, the apparatus includes an intramedullary nail, which is configured for insertion inside a medullary cavity of the bone. The transducer is mounted within the intramedullary nail in proximity to a fixation hole in the intramedullary nail, so as to engage the inner wall of the medullary cavity at the selected location in alignment with the fixation hole.

In other embodiments, the apparatus includes an elongate shaft configured for insertion into the cavity, wherein the transducer is fixed at the distal end of the shaft.

In some embodiments, the apparatus includes an acoustic probe, which is configured to be applied to a surface of the body in proximity to the bone, and to output a detection signal indicative of acoustical modulation due to the movement of the bone. A processor is configured to generate and output an indication of the location responsively to the detection signal. In a disclosed embodiment, the acoustic probe includes an ultrasound transducer, which is configured to direct ultrasonic waves toward the bone and to detect the acoustical modulation as a Doppler shift of the ultrasonic waves. Additionally or alternatively, the processor is configured to indicate, responsively to the detection signal, a position and direction for application of a surgical tool to the bone in order to create a hole through the bone at the location.

There is also provided, in accordance with an embodiment of the invention, a method for localization, which includes bringing a transducer into engagement with a surface of a wall of a cavity inside a body of a living subject. The transducer is driven so as to cause an echogenic movement of the wall at a location of the transducer. An acoustical modulation due to the movement of the wall is detected, in order to generate and output an indication of the location responsively to the detected acoustical modulation.

In some embodiments, detecting the acoustical modulation includes applying an acoustic probe to a surface of the body in proximity to the wall, and outputting from the acoustic probe a detection signal indicative of acoustical modulation due to the movement of the wall. In a disclosed embodiment, the acoustic probe includes an ultrasound transducer, and detecting the acoustical modulation includes directing ultrasonic waves from the ultrasound transducer toward the wall, and detecting the acoustical modulation as a Doppler shift of the ultrasonic waves. Additionally or alternatively, outputting the indication includes indicating, responsively to the detection signal, a position and direction for application of a surgical tool to the wall in order to create a hole through the wall at the location.

In some embodiments, bringing the transducer into engagement includes contacting the surface of a bone within the body, and driving the transducer causes the bone to vibrate. In a disclosed embodiment, the method includes inserting an intramedullary nail inside a medullary cavity of the bone, wherein bringing the transducer into engagement includes placing the transducer within the intramedullary nail in proximity to a fixation hole in the intramedullary nail, so as to engage an inner wall of the medullary cavity at the location of the transducer in alignment with the fixation hole.

There is additionally provided, in accordance with an embodiment of the invention, a method for locating a target portion of an intracorporeal tissue layer in a living subject from an extracorporeal location. The method includes deforming the target portion relative to a surrounding portion of the intracorporeal tissue layer by driving a pusher head against the target portion, thereby generating a distinguishable acoustic signal. A carrier wave is recorded at the extracorporeal location, and a demodulator is applied to extract the distinguishable acoustic signal from the recorded carrier wave.

In a disclosed embodiment, at least one of the distinguishable acoustic signal and the recorded carrier wave is analyzed in order to determine a disposition of the target portion relative to the extracorporeal location.

Additionally or alternatively, the method includes repeating the deforming until the distinguishable acoustic signal is generated or detected.

Further additionally or alternatively, the method includes generating an acoustic wave at the extracorporeal location, wherein the carrier wave is generated by reflection of the acoustic wave from a vicinity of the target portion.

Alternatively, the carrier wave is generated by the deforming.

In a disclosed embodiment, the pusher head engages the target portion via a pusher distal contact surface being equal or smaller in size than the target portion.

Optionally, the pusher head engages a first side of the intracorporeal tissue layer, and the carrier wave is generated on a second side of the intracorporeal tissue layer, opposite the first side.

In some embodiments, the pusher head is included in a pusher operatively connected to at least one of a motion generator and a signal generator.

Alternatively, deforming the target portion includes applying a transducer to the target portion. In some embodiments, the transducer includes at least one of a piezoelectric crystal and a mechanical vibrator.

In a disclosed embodiment, the deforming includes reciprocating movements of the target portion relative to the surrounding portion. Optionally, the reciprocating movements include vibrational movement.

In some embodiments, deforming the target portion includes driving the pusher head at a frequency no greater than 1 kHz, or alternatively at a frequency between 1 kHz and 100 kHz, or at a frequency between 100 kHz and 1 MHz, or at a frequency between 1 MHz and 10 MHz.

In one embodiment, the pusher head is fixated to the target portion prior to the deforming. Alternatively or additionally, the pusher head presses against the target portion throughout the deforming.

Optionally, the recording is performed using an ultrasound probe, and applying the demodulator includes receiving a signal from at least one of an ultrasound system and a Doppler system.

In some embodiments, the intracorporeal tissue layer is part of a bone, such as a skull, a vertebra, and a long bone. In another embodiment, the intracorporeal tissue layer is part of a blood vessel wall.

There is further provided, in accordance with an embodiment of the invention, an implant including an implant body sized to fit in a bodily organ of a living subject surrounded by a bodily wall, and a rigid pusher with a pusher head selectively extendable from the implant body for engaging a target portion of the bodily wall. A motion generator is operatively connected to the pusher and configured for driving the pusher head against the target portion so as to deform the target portion relative to a surrounding portion of the bodily wall sufficiently to generate a distinguishable acoustic signal.

In the disclosed embodiments, the bodily wall is selected from a set of bodily walls consisting of a bone tissue, a cartilage tissue, a tooth and a connective tissue.

In some embodiments, the motion generator includes at least one ultrasonic vibration actuator, such as a piezoelectric element.

In a disclosed embodiment, the implant includes a coupling mechanism configured for at least one of fixating the pusher head to the target portion and continuously pressing the pusher head against the target portion, on a first side of the bodily wall.

In some embodiments, a signal generator is operatively connectable to the motion generator and configured to activate the motion generator to drive the probe head in accordance with a preset pattern. In a disclosed embodiment, the implant includes an amplifier connecting between the signal generator and the motion generator and configured to amplify signals generated by the signal generator. In some embodiments, the maximal amplified signal producible through the amplifier is less than 10 W, or between 10 W and 200 W.

In a disclosed embodiment, at least one of the pusher and the motion generator is configured for generating at least one of a longitudinal deformation and a shear deformation of the target portion relative to the surrounding portion of the bodily wall.

There is moreover provided, in accordance with an embodiment of the invention, a method for fixating an implant in a bone. The method includes inserting the implant in a cavity of the bone and using the implant, positioning a motion generator to engage a target portion of a bone wall surrounding the cavity in proximity to an anchoring portion of the bone wall. The motion generator is activated to deform the target portion relative to a surrounding portion of the bodily wall sufficiently to generate a distinguishable acoustic signal beyond the bone wall. At an extracorporeal location, an imaging device is applied for detecting the distinguishable acoustic signal. Based on the detected acoustic signal, a disposition of the target portion relative to the extracorporeal location is determined. The bone wall is penetrated with a fixating member at the anchoring portion. The fixating member is connected to the anchoring portion, thereby fixating the implant in the bone.

In some embodiments, detecting the distinguishable acoustic signal includes measuring at least one parameter associated with a deformation of the target portion, selected from a set of parameters consisting of frequency, echogenicity, amplitude, velocity, acceleration, temperature, elasticity and ductility.

Optionally, penetrating the bone wall is preceded by drilling the anchoring portion of the bone wall.

In some embodiments, the implant includes at least one transverse opening sized and shaped to accommodate the fixating member passing therethrough, wherein determining the disposition includes positioning the transverse opening to align with the anchoring portion. In one such embodiment, positioning the motion generator includes passing the motion generator through a lumen in the implant from and into a chosen alignment with the transverse opening.

There is furthermore provided, in accordance with an embodiment of the invention, a system for fixating a long bone. The system includes an intramedullary nail configured for insertion in a cavity of the long bone. A motion generator is positioned in the intramedullary nail in proximity to a fixation opening of the intramedullary nail and configured for effecting reciprocal deformations of a target portion in a bone wall surrounding the cavity relative to a surrounding portion of the bone wall.

In some embodiments, the system includes a nail fixator template fixedly connectable with a first end thereof to a proximal end of the intramedullary nail. The template incorporates at least one directional passage sized and configured for aligning a nail fixator in a chosen spatial direction relative to the fixation opening when fixedly connected to the proximal end of the intramedullary nail. In a disclosed embodiment, the template includes means to align the directional passage with the chosen spatial direction. Additionally or alternatively, the template includes a holder for holding and directing an ultrasound probe, and the holder may include the directional passage. Further alternatively, the ultrasound probe includes the directional passage.

There is also provided, in accordance with an embodiment of the invention, a method for fixating an intramedullary nail in a cavity of a long bone. The method includes positioning a motion generator in proximity to a fixation opening of the intramedullary nail within the cavity in alignment with a target portion in a bone wall surrounding the cavity. A first end of a nail fixator template is attached to a proximal end of the intramedullary nail. The template incorporates at least one directional passage sized and configured for aligning a nail fixator therethrough. The motion generator is actuated so as to deform the target portion relative to surrounding portion of the bodily wall sufficiently to generate a distinguishable acoustic signal beyond the bone wall. The distinguishable acoustic signal is detected using an imaging device at an extracorporeal location. A disposition of the target portion is determined relative to the extracorporeal location. The directional passage is adjusted, using the determined disposition, to align in a chosen spatial direction relative to the fixation opening.

In some embodiments, the method includes creating a transcutaneous passage in soft tissue in proximity to the long bone in alignment with the directional passage, and drilling a hole through the bone wall across the long bone in a vicinity of the target portion. A nail fixator is delivered through the hole and fixating the nail fixator to at least one of the intramedullary nail and the bone wall.

The present invention will be more fully understood from the following detailed description of the embodiments thereof, taken together with the drawings in which:

### BRIEF DESCRIPTION OF THE DRAWINGS

FIG. 1 is schematic pictorial illustration of a distal targeting system, in accordance with an embodiment of the invention;

FIG. 2A is a schematic sectional illustration of a bone into which an intramedullary nail with an internal vibrating device has been inserted, in accordance with an embodiment of the invention;

FIG. 2B is a schematic sectional illustration of the bone of FIG. 2A, showing operation of the internal vibrating device in finding the location of a fixation hole in the intramedullary nail, in accordance with an embodiment of the invention;

FIG. 3 is a schematic reproduction of an ultrasound image showing an indication of the location of an internal vibrating device in a bone, in accordance with an embodiment of the invention;

FIG. 4 is a schematic sectional illustration of the bone of FIGS. 2A and 2B, showing drilling of a hole through the bone under guidance of the ultrasonically-indicated location of the fixation hole, in accordance with an embodiment of the invention;

FIG. 5 is a block diagram that schematically illustrates an implantation system, in accordance with another embodiment of the invention;

FIG. 6 is a block diagram that schematically shows further details of the system of FIG. 5, in accordance with an embodiment of the invention;

FIG. 7 is a block diagram that schematically illustrates an implantation system, in accordance with an alternative embodiment of the invention;

FIG. 8 is a block diagram that schematically illustrates an implantation system, in accordance with yet another embodiment of the invention;

FIG. 9 is a schematic sectional view of a system for locating a target portion of an intracorporeal tissue layer from an extracorporeal location, in accordance with an embodiment of the invention;

FIG. 10 is a schematic sectional view of a catheterization system, in accordance with another embodiment of the invention;

FIG. 11 is a schematic sectional view of a cranial implantation system, in accordance with yet another embodiment of the invention; and

FIG. 12 is a schematic section illustration of a system for fixating an intramedullary nail in a medullary cavity of a bone, in accordance with an embodiment of the invention.

### DETAILED DESCRIPTION OF EMBODIMENTS

Current medical practice involves numerous means and technologies for marking or locating invasive medical instrumentation using non-invasive imaging means, such as X-ray imaging. X-ray technology is advantageous since that it can be used effectively in imaging all types of bodily tissues in the subject (“intracorporeal tissue”) from outside the body (“extracorporeal location”). By contrast, ultrasound, for example, cannot be used effectively for imaging relatively thick and/or dense bone tissue layers, or other tissues located beyond the bone, relative to the ultrasound probe in use. Due to the expanding use of X-ray imaging (including fluoroscopy, CT and other techniques) for guiding minimally invasive procedures, there is a growing effort to develop effective means that will replace or at least diminish use of X-rays, in order to decrease exposure of patients and medical teams to X-ray radiation, which is associated with carcinogenesis and other adverse effects. Excessive use of contrast enhancing media (e.g., iodine-based chemicals) during fluoroscopy is also considered harmful to the patient.

Numerous medical apparatuses are designed for use under X-ray imaging, including implants (e.g., orthopedic implants, stents, artificial machines, electrodes, and leads) and delivery devices for implants or drugs (e.g., catheters, needles, and ports), for example.

Embodiments of the present invention that are described herein provide improved methods and apparatus for identifying a location inside the body of a living subject using acoustical detection, for example by an ultrasonic probe in contact with the body surface. In the disclosed methods, a transducer is brought into engagement with a surface of the wall of a cavity inside the body and is driven so as to cause a vibrational movement of the wall at the location of the transducer. A processor detects an acoustical modulation that occurs due to the vibrational movement of the wall and thus generates an indication of the location based on ultrasound echoes and/or Doppler imaging, for example.

Methods and apparatus in accordance with some embodiments of the invention are useful particularly in orthopedic applications, such as distal targeting of the location where a hole should be drilled through a bone. In this context, the disclosed embodiments provide a reliable indication of the position and direction for application of a surgical tool to the bone in order to drill the hole through the bone, while reducing substantially the need for X-ray imaging. Alternatively, however, the principles of the present invention may be applied, mutatis mutandis, to other body cavities having elastic walls, such as arteries and chambers of the heart, as well as body walls made of cartilage or connective tissue.

For purposes of generating the desired vibrational movement, some embodiments of the present invention provide an invasive medical device comprising an elongate shaft for insertion into a cavity inside a bone, with a transducer fixed at the distal end of the shaft and configured to contact the inner wall of the cavity at a selected location. The shaft may be either rigid or flexible. The transducer may comprise, for example, a piezoelectric crystal or a mechanical vibrator. As another example, the transducer may apply pulses of thermal energy to the inner wall, causing local deformation of the targeted bone wall portion. In some embodiments, a drive circuit applies a signal to the transducer so as to cause a vibrational movement of the bone at the selected location.

In other embodiments, the transducer (or multiple transducers) is inserted into the cavity without the use of a shaft in doing so. For example, one or more transducers may be pre-installed in a surgical appliance, such as an intramedullary nail, which is then inserted inside a medullary cavity of the bone. The transducer or transducers are mounted within the intramedullary nail in proximity to fixation holes in the intramedullary nail, possibly protruding through these fixation holes, and thus engage the inner wall of the medullary cavity at locations that are aligned with the fixation holes.

While the transducer (whether or not attached to a shaft) is driven to cause vibrational movement, an acoustic probe, such as an ultrasound transducer is applied to the body surface in proximity to the bone, and outputs a signal to the processor that is indicative of the acoustical modulation due to the vibrational movement of the bone. In one embodiment, the probe directs ultrasonic waves toward the bone and detects the acoustical modulation as a Doppler shift of the ultrasonic waves.

In the embodiments that are described in detail hereinbelow, a vibrational device is inserted inside the bore of an intramedullary nail, which is inserted into a medullary cavity of a fractured bone that is undergoing surgery. The transducer is configured to protrude from the shaft through one of the fixation holes in the intramedullary nail, and thus contacts and causes vibration of the inner wall of the medullary cavity at a location that is closely aligned with the fixation holes of both sides of the nail. Optionally, the transducer may additionally be configured to thin the bone at the contact location.

The above features enable positive, reliable alignment of surgical tools, such as a bone drill, while minimizing the need for X-ray exposure. They may be used not only in distal targeting for fixation of intramedullary appliances, but also in other surgical applications, for example in drilling through the cranium for insertion of shunts and other sorts of implants.

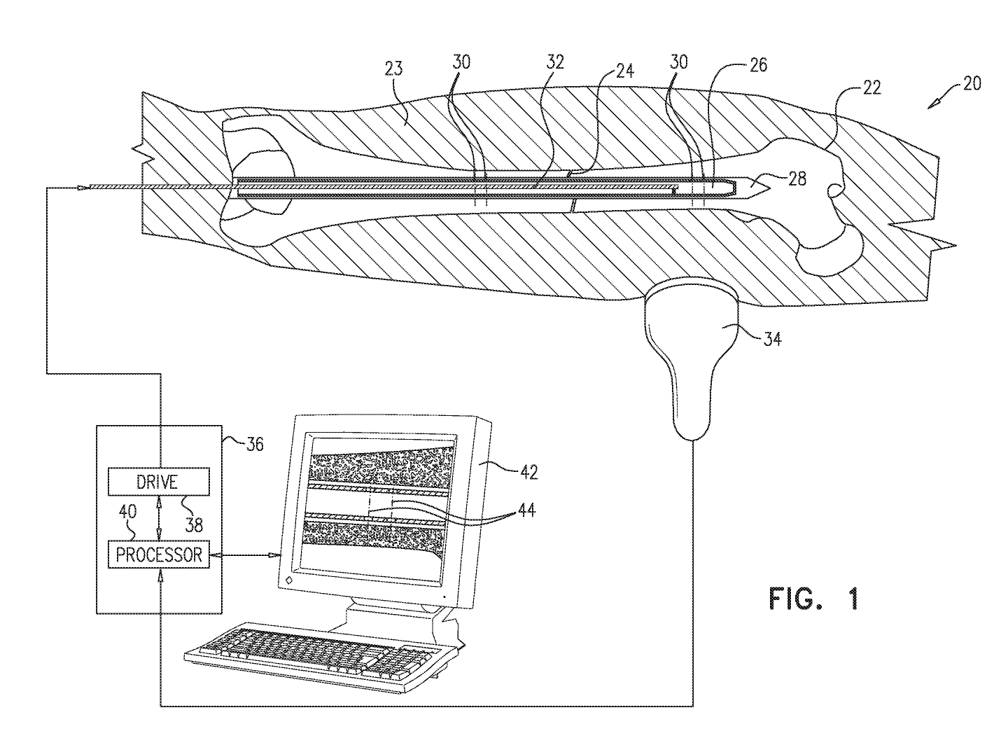

FIG. 1 is schematic pictorial illustration of a distal targeting system 20 based on ultrasonic detection, in accordance with an embodiment of the invention. In the pictured embodiment, system 20 is applied in repair of a fracture 24 in a bone 22 within a leg 23 of a subject, for example, in the femur (as shown in the figure) or alternatively, the tibia or any other long bone that can be treated in this manner. At the stage of the procedure that is shown in FIG. 1, the surgeon has drilled an opening into a medullary cavity 28 of bone 22 and has inserted an intramedullary nail 26 into the cavity. As the next step in the procedure, the surgeon must drill holes through bone 22 aligned with the locations of fixation holes 30 in nail 26, in order to drive screws through the holes and thus secure the nail in place.

In order visualize the locations of holes 30, a vibrational device 32 is inserted into the central bore of intramedullary nail 26 and contacts the inner wall of cavity 28. Details of this device and its operation are shown in the figures that follow. Device 32 may be inserted into nail 26 either before or after insertion of nail 26 into medullary cavity 28, and in the former case may also be supplied to the surgeon as a pre-installed accessory together with nail 26. An acoustic probe 34, comprising an ultrasound transducer as is known in the art, is applied to the surface of leg 23 in proximity to bone 22, and specifically in proximity to and/or directed towards the one of holes 30 whose location is to be targeted.

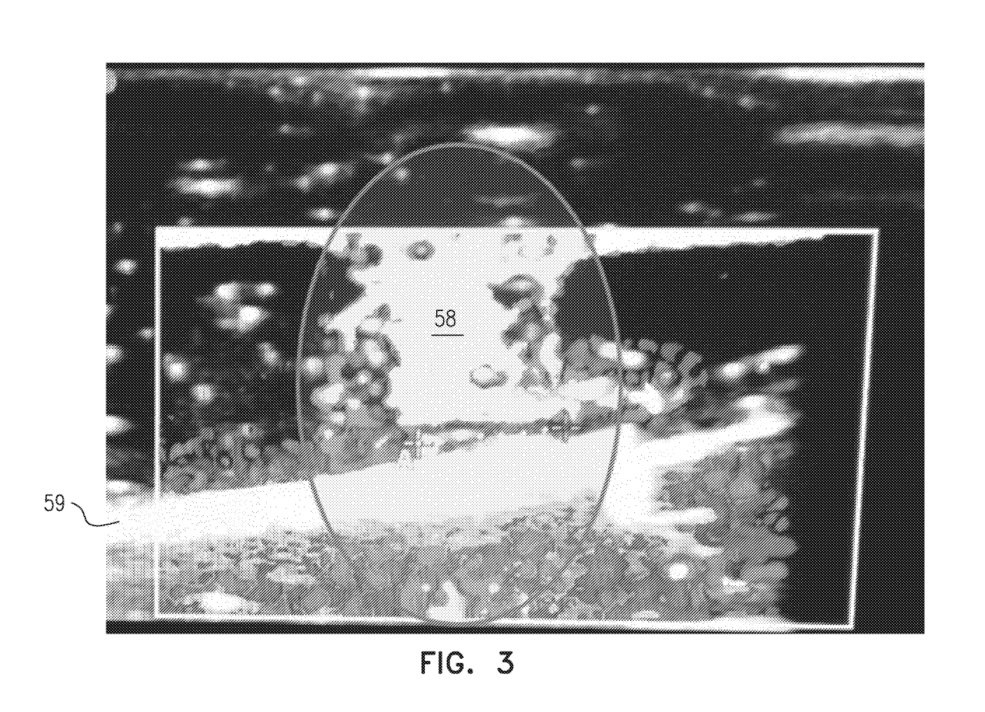

System 20 comprises a console 36, including a drive circuit 38 and a processor 40. Drive circuit 38 applies a drive signal to device 32, which causes a local vibrational movement of bone 22 at the location of fixation hole 30. This vibrational movement gives rise to an echogenic disturbance, causing an acoustical modulation that is detectable by probe 34. In the example illustrated below in FIG. 3, this modulation is observed as a Doppler shift in ultrasonic waves that are emitted by and reflected back to probe 34. Alternatively, probe 34 may directly detect acoustic waves emitted from bone 22 at the frequency of vibration of device 32.

Probe 34 outputs a detection signal that is indicative of the detected acoustical modulation to processor 40. Additionally or alternatively, probe 34 may be connected to an imaging system (not shown), such as a portable ultrasound system, optionally with Doppler ultrasound capabilities. In some embodiments, processor 40 comprises a general-purpose computer processor, which is programmed in software to carry out the functions that are described herein. This software may be downloaded to processor 40 in electronic form, or it may, alternatively or additionally, be stored on tangible, non-transitory computer-readable media, such as optical, magnetic, or electronic memory media. Further alternatively or additionally, at least some of the functions of processor 40 may be implemented in hard-wired or programmable logic circuits.

In some embodiments, based on the detection signal from probe 34 or from an imaging system to which the probe is connected, processor 40 generates and outputs an indication of the location of the vibrating transducer at the distal end of device 32, and hence indicating accurately the location of fixation hole 30. For example, in some embodiments, the echogenic disturbance created by device 32 results in the appearance of an artifact in an ultrasound image appearing on a display screen 42, which indicates the location to the medical practitioner. Display screen 42 may be a part of the imaging system mentioned above or an independent part of system 20. Alternatively or additionally, processor 40 analyzes the image in order to compute the target location. The location may be computed, for example, by moving probe 34 systematically along the surface of leg 23, measuring the distance to the source of the acoustical modulation at different positions of the probe, and triangulating the measurements in order to find the source of the acoustical modulation. Additionally or alternatively, probe 34 may comprise a directional detector, such as a phased array, as is known in the art, which is gated and swept in order to find both the distance and angle between the probe and the location of hole 30.

In some embodiments, the imaged artifact can be used by the surgeon in calculating and determining the entry point on the patient's skin and the drilling path, from the entry point to hole 30, as required for spatial alignment with the two corresponding holes 30 of both sides of nail 26. Calculation and/or determination of the entry point and drilling path may be performed by the surgeon himself, optionally assisted by other means, such as with information provided by the imaging system mentioned above.

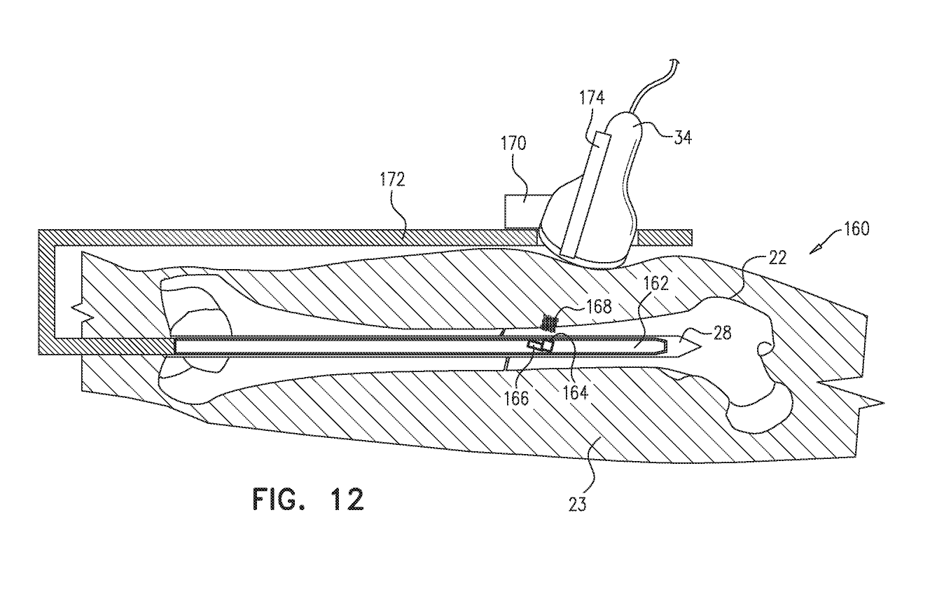

In other embodiments, processor 40 outputs the location indication to an output device, for either manual use by the surgeon or automated guidance in drilling a hole through bone 22 in order to engage fixation hole 30 (as illustrated in FIG. 4). In the example shown in FIG. 1, processor 40 outputs the location indication to display screen 42, where the location indication takes the form of an axis 44, defining the location and orientation along which the drill should be directed through bone 22 (or multiple axes 44 for multiple fixation holes). Alternatively, probe 34 may be mounted on a stereotactic frame (as shown in FIG. 12, for example) together with the drill, in which case processor 40 can automatically or semi-automatically control the position and orientation of the drill based on the output of the probe, or the surgeon may control the position and orientation manually based on the image on the display screen, as described above.

In some other embodiments, probe 34 is connected to an imaging system, and both are unconnected with and/or operate independently of system 20. In this case, the means for generating an echogenic disturbance are separately controlled, and the echogenic disturbance is generated independently of the imaging means.

The generated echogenic disturbance may possess specific characteristics in order to facilitate an accurately distinguishable artifact on screen. As will be further detailed below, means may be used for generating local reciprocal deformation to a target portion on wall of bone 22 adjacent fixation holes 30, optionally directly in front of a particular fixation hole 30. This local deformation of the target portion, relative to its surrounding bone portion, is configured with significant echogenicity, which can be picked up by sonographic means (e.g., ultrasound, color Doppler, continuous-wave Doppler, pulsed-wave Doppler, or other means) and be accurately distinguishable as an artifact in the imaging product or image screen. In some embodiments, the difference in frequency and/or in amplitude of the reciprocally deforming (e.g., vibrating) target portion is substantial, so that the generated artifact is relatively small in size (e.g., 5 mm or less in diameter) and visually identifiable and bordered relative to its surroundings.

The target portion on bone 22 is optionally similar in size to, or smaller than, the size of fixation hole 30. In some embodiments, the deformed area occupied by the target portion is about 10 mm or less, optionally 5 mm or less, or possibly 1 mm or less, in diameter. Local deformation, in terms of frequency and/or amplitude, may be determined according to the type of intracorporeal tissue layer that comprises the target portion and its surrounding portion, in this example bone 22. For more elastic and/or less ductile tissue types, such as soft tissues, there may be a need for increased amplitude (e.g., oscillation pattern or stroke length) and decreased frequency, relative to calcified tissues such as bones, for example, which may require higher frequencies, such as from within the ultrasonic range. For soft tissues, applicable frequencies may be 1 kHz or less, optionally 100 Hz or less, or optionally 1 Hz or less, with stroke length of about 0.1 mm to about 10 mm, or about 0.2 mm to about 2 mm, for example. For hard tissues, chosen frequencies may be 10 MHz or less, optionally about 1 MHz or less, or optionally about 100 KHz or less, for example, with stroke lengths of about 10 to 1,000 microns, optionally 50 to 100 microns. Greater oscillations or stroke lengths, optionally with increased stroke forces (10 gr or more, optionally 100 gr or more), can be used when local damage to the tissue is permitted, such by thinning or drilling through the target portion in the process of its deforming or vibrating.

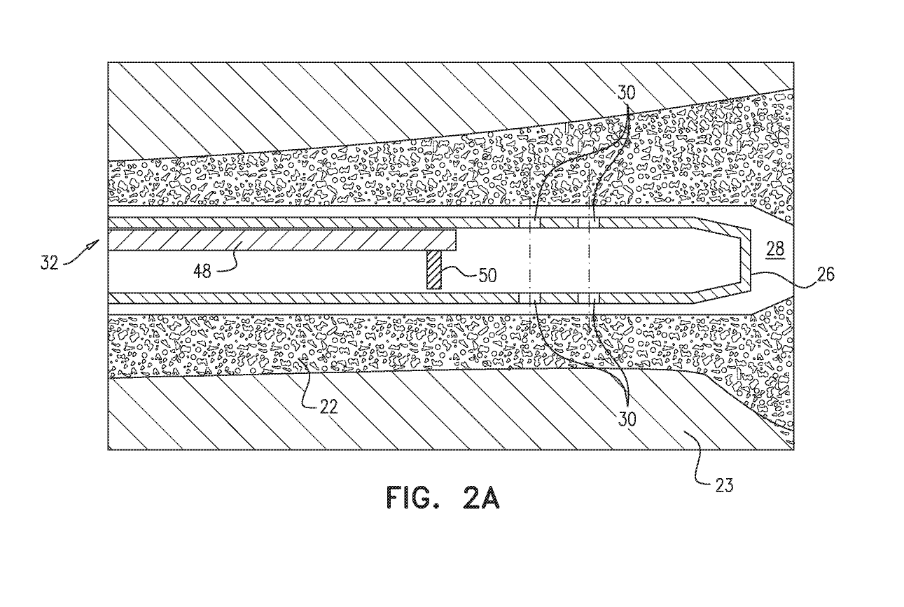

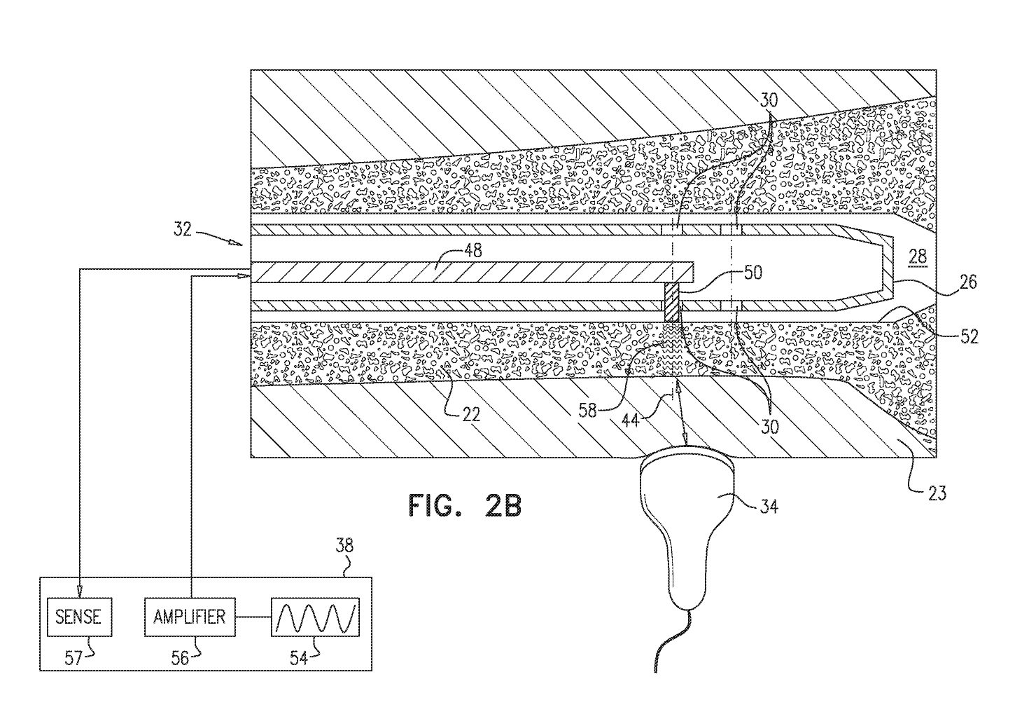

Reference is now made to FIGS. 2A and 2B, which are schematic sectional illustrations showing details of device 32 in use inside bone 22, in accordance with an embodiment of the invention. FIG. 2A shows a preliminary stage as device 32 is being advanced through the central bore of nail 26 toward the location of fixation holes 30, while FIG. 2B shows the device in use in localizing one of the fixation holes.

Device 32 comprises an elongate shaft 48, which is fitted inside the bore of nail 26, with a transducer 50 fixed at the distal end of the shaft. In the pictured embodiment, shaft 48 comprises a rigid rod, to which transducer 50 is attached and which thus permits the operator to advance the transducer through the bore of nail 26, as illustrated in FIG. 2A. Transducer 50 is shaped and sized to fit through any of fixation holes 30 (which are typically about 1 cm in diameter). Transducer 50 may include a rigid pusher that is configured to oscillate longitudinally through a fixation hole 30, with a magnitude, amplitude and/or frequency sufficient to generate an effective echogenic disturbance via a target portion of bone 22 that it engages, as described above. Thus, upon reaching the desired location, transducer 50 protrudes through hole 30 and engages an inner wall 52 of medullary cavity 28, as shown in FIG. 2B. The rigidity of shaft 48 also enables the operator or an automated actuator (not shown) to control the pressure applied by transducer 50 against inner wall 52.

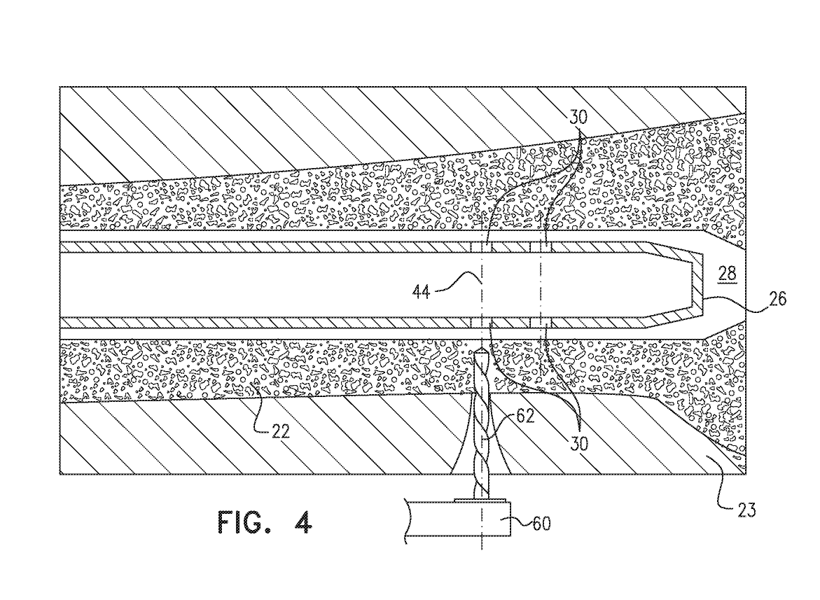

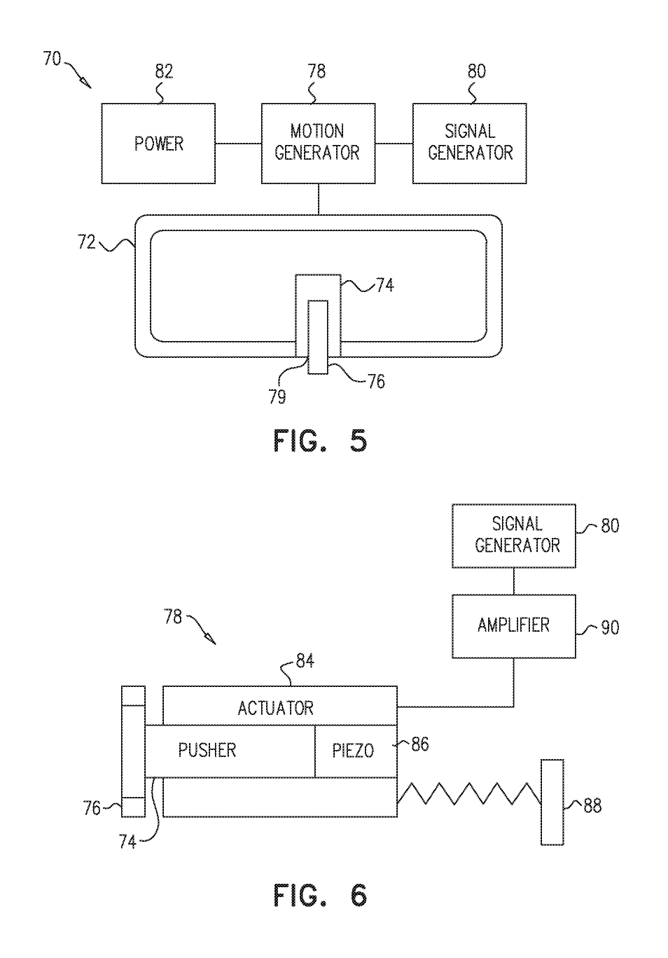

Alternatively, as noted earlier, device 32 may be pre-installed inside nail 26 in the location shown in FIG. 2B. In this case, shaft 48 may be either rigid or flexible, and multiple transducers may be pre-installed within or in alignment with holes 30. The flexible shaft provides power to and controls transducer 50, and may also be used to withdrawn the transducer from nail 26 when device 32 is no longer needed. Alternatively, and similarly to as shown in FIG. 5 for example, the transducers may be permanently installed in nail 26 in proximity to holes 30, with suitable electrical connections for driving the transducers but without a shaft or other means for moving the transducers within the nail.

Further alternatively, any other suitable means may be applied to fix and hold transducer 50 in the appropriate location relative to hole 30 and inner wall 52 of bone 22 (such as a coupling mechanism 88 that is shown in FIG. 6, for example). For example, transducer 50 may be contained in or attached to a balloon, which is inflated with a suitable fluid, such as saline solution, in order to anchor the transducer in place.

Transducer 50 may comprise any suitable means for imparting local vibrational movement to bone 22. For example, transducer 50 may comprise a piezoelectric crystal or a mechanical vibrator. Optional frequencies may be in the range between 1 and 100 kHz, or possibly in the range between 10 and 50 kHz. Alternatively, transducers comprising piezoelectric crystals may be driven to apply vibrations at higher frequencies, for example up to 1 MHz, or even up to 10 MHz. Optionally, a frequency of vibration is chosen at which bone 22 has a strong vibrational response, so that probe 34 will observe a strong acoustical modulation due to the local deformation of the bone. In one embodiment, transducer 50 comprises a phased array of piezoelectric crystals, which are controlled by driver 38 to apply vibrational energy directionally to bone 22.

In an alternative embodiment, transducer 50 is configured to apply pulses of thermal energy to bone 22, which thus cause the bone to vibrate at the pulse frequency. For this purpose, for example, transducer 50 may comprise a pulsed infrared or visible laser radiation source or a radio-frequency (RF) radiation source. Absorption of the radiation in or near wall 52 of bone 22 causes local vibrations at the pulse frequency, for example due to cavitation of fluid within medullary cavity 28.

Although FIG. 2B shows the tip of transducer 50 (e.g., a pusher head) in actual contact with inner wall 52 of bone 22, this contact need not be continuous during actuation of the transducer. Thus, depending on the pressure applied on transducer 50 through shaft 48, the transducer may press continuously against wall 52 or it may tap against the bone surface at the frequency of vibration, without continuous contact. Alternatively, transducer 50 may engage inner wall 52 without direct physical contact, for example by directing pulses of acoustical or thermal energy toward the selected location on wall 52. In some embodiments, transducer 50 has a tip or pusher head sized and configured for contacting and deforming a target portion of bone 22 that maximizes the size of the generated artifact on screen. Optional pusher head diameters may be in the range between 0.1 and 10 mm, or optionally in the range between 0.5 and 5 mm.

As illustrated in FIG. 2B, driver 38 comprises a signal generator 54, which generates a driving waveform at the desired vibrational frequency of transducer 50, and a power amplifier 56, which amplifies and applies a corresponding drive signal to the transducer. Transducer 50 transfers the energy of the drive signal to bone 22, thus giving rise to a local vibrational movement in an area 58 of the bone that is engaged by the transducer. Optionally, a sensing circuit 57 measures properties such as the force or pressure exerted by transducer 50 against wall 52.

In some embodiments, system 20 communicates with acoustic probe 34 via processor 40, whereby acoustic probe 34, when positioned against the outer surface (skin) of leg 23, can be applied for outputting a detection signal that is indicative of the acoustical modulation generated due to the vibrational movement of area 58 of bone 22. In some such embodiments, processor 40 (FIG. 1) can be programmed to analyze this signal in order to identify area 58 and thus find the position and orientation of axis 44. In addition, processor 40 can use the detection signal from probe 34, possibly together with the output of sensing circuit 57, in controlling the operation of device 32. For example, processor 40 may vary the frequency of signal generator 54 and/or the gain of amplifier 56 in order to find a combination of frequency and amplitude that causes strong vibrational movement of bone 22 and hence marked acoustical modulation. Additionally or alternatively, processor 40 may control the pressure of transducer 50 against wall 52 (either automatically or by outputting instructions to the system operator) to ensure efficient transfer of vibrational energy from the transducer to the bone.

In an alternative embodiment, driver 38 applies sufficient energy to transducer 50 so that the mechanical or thermal pulses applied to inner wall 52 of bone 22 not only vibrate the bone, but also erode away at least a part of the inner wall. Consequently, bone 22 is thinned in this location, thus facilitating stronger vibrational movement of the bone and possibly even creating a guide hole through the bone wall for subsequent drilling. Possible erosion or drilling may be only partial to an extent sufficient to form a local acoustic window, thus facilitating increased penetrability to ultrasonic waveforms. Alternatively or additionally, the erosion or drilling may be sufficient to change the mechanical characteristics of the target portion in a way that reduces its resistance to deformation and/or vibration relative to surrounding portion of bone 22.

FIG. 3 is a schematic reproduction of an ultrasound image showing an indication of the location of an internal vibrating device in a bone, in accordance with an embodiment of the invention. This figure is based on an actual Doppler ultrasound image, taken of a bone with a vibrating needle (serving as transducer 50) placed against the inner wall of the medullary cavity. The needle was driven to vibrate at a frequency of about 33 kHz, and Doppler image signals were obtained from an ultrasound probe operating in the range of 6-13 MHz.

As can be seen in FIG. 3, a strong Doppler shift was observed in area 58, in proximity to the vibrating needle and adjacent to an area 59 of the image corresponding to the bone wall. The Doppler signal observed in the figure is due to the Doppler shift of the ultrasonic probe signal arising from the vibrational velocity of the bone in area 58. The width of area 58 observed in this Doppler image was about 0.5 cm.

FIG. 4 is a schematic sectional illustration of bone 22, showing drilling of a hole through the bone under guidance of the ultrasonically-indicated location of fixation hole 30, in accordance with an embodiment of the invention. In this example, after identifying axis 44 as illustrated in the preceding figures, device 32, including transducer 50, has been withdrawn from nail 26. Alternatively, transducer 50 may be left in place. A surgical tool, such as a drill 60, is positioned and oriented so that a bit 62 of the drill is aligned with axis 44. The drill is then actuated to drill through bone and thus create an opening in the bone through which a fixation screw can be inserted.

FIG. 5 is a block diagram that schematically illustrates an implantation system 70, in accordance with another embodiment of the invention. System 70 comprises an implant body 72 sized to fit in a bodily organ of a living subject surrounded with a bodily wall. A rigid pusher 74 comprises a pusher head 76 selectively extendable from the implant body for engaging a target portion of the bodily wall. (Pusher 74 can be considered a type of transducer, as defined above.) A motion generator 78 is operatively connected to pusher 74 and configured for driving pusher head 76 through an opening 79 in implant body 72 against the target portion, with a chosen magnitude and/or frequency sufficient for deforming the target portion relative to a surrounding portion of the bodily wall so as to generate a distinguishable acoustic signal. Pusher 74 and/or motion generator 78 is configured for generating longitudinal deformation and/or shear deformation of the target portion relative to the surrounding portion of the bodily wall. The bodily wall can be, for example, a bone tissue, a cartilage tissue, a tooth, a blood vessel wall, or soft tissue (e.g., a connective tissue). A signal generator 80 inputs a driving signal to motion generator 78, while a power supply 82 provides the required electrical power.

FIG. 6 is a block diagram that schematically shows further details of the system of FIG. 5, in accordance with an embodiment of the invention. In this embodiment, motion generator 78 includes at least one ultrasonic vibrational actuator 84, which may include, for example, a piezoelectric element 86 or alternatively, a mechanical vibrator. Additionally or alternatively, system 70 comprises a coupling mechanism 88 configured for fixating pusher head 76 to the target portion, or to continuously press against the target portion, at a first side of the bodily wall.

Signal generator 80 activates motion generator 78 to drive pusher 74 in accordance with a preset pattern. In the pictured embodiment, an amplifier 90 is connected between signal generator 80 and motion generator 78 and amplifies the signals generated by the signal generator. In one embodiment, the maximal amplified signal producible through the amplifier is less than 10 W. In another embodiment, the maximal amplified signal producible through the amplifier is between 10 W and 200 W. In one embodiment, pusher 74 is configured to oscillate and/or move pusher head 76 reciprocally in and out through opening 79.

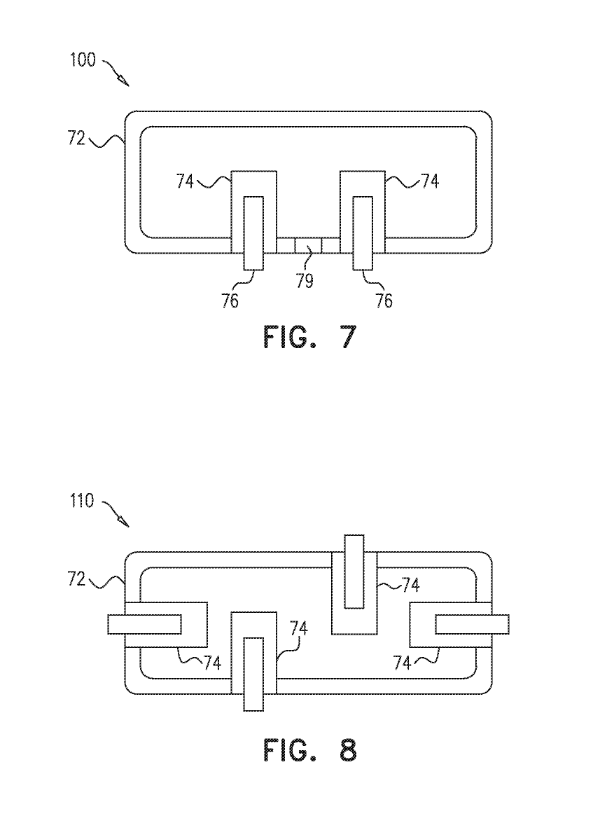

FIG. 7 is a block diagram that schematically illustrates an implantation system 100, in accordance with an alternative embodiment of the invention. In this case, pushers 74 are located alongside opening 79 rather than actually protruding through the opening as in the preceding embodiment. The terms “adjacent” and “in proximity” are used in this context, in the present description and in the claims, to cover this range of possible locations of the pushers, including both protrusion through and positioning alongside an opening in implant body 72.

FIG. 8 is a block diagram that schematically illustrates an implantation system 110, in accordance with yet another embodiment of the invention. This embodiment illustrates that pushers 74 need not be located only on one side of implant body, but may rather be located on two or more sides, depending on application requirements.

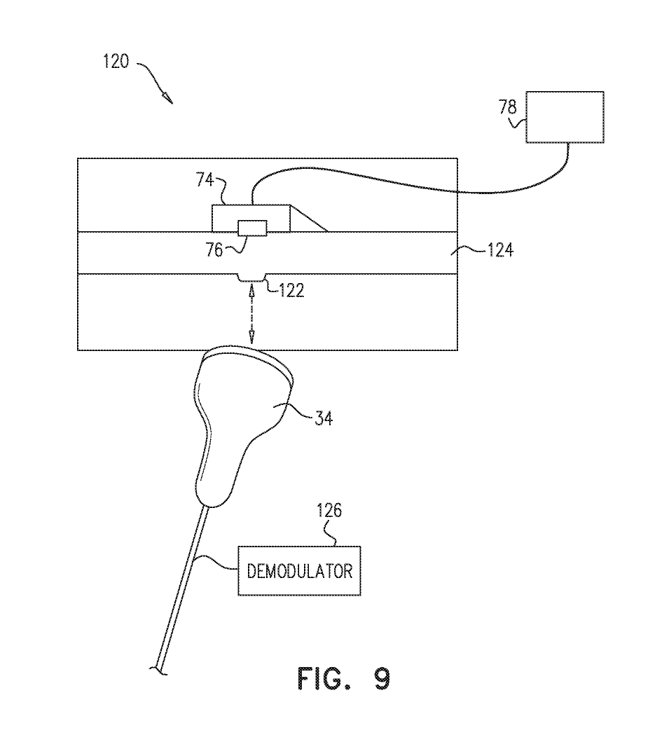

FIG. 9 is a schematic sectional view of a system 120 for locating a target portion 122 of an intracorporeal tissue layer 124 from an extracorporeal location, in a living subject, in accordance with an embodiment of the invention. Target portion 122 is deformed relative to the surrounding portion of intracorporeal tissue layer 124 by driving pusher head 76 against the target portion, with a chosen magnitude and/or frequency, thereby generating a distinguishable acoustic signal. The distal contact surface of pusher head 76 is optionally equal to or smaller in size than the target portion, optionally about 10 mm or less, or about 5 mm or less, or about 1 mm or less, in diameter. Probe 34 records a carrier wave at its extracorporeal location. A demodulator 126 extracts the distinguishable acoustic signal from the recorded carrier wave.

In some embodiments, processor 40 (FIG. 1) analyzes the distinguishable acoustic signal and/or the recorded carrier wave in order to determine the disposition, i.e., the distance and/or direction, of target portion 122 relative to the extracorporeal location. In some embodiments, probe 34 generates an acoustic wave at the extracorporeal location, and the carrier wave is generated by the acoustic wave reflecting from target portion 122 and/or the surrounding portion. Alternatively, the carrier wave is generated by the deforming.

In the embodiment shown in FIG. 9, pusher head 76 engages a first side of intracorporeal tissue layer 124, while the carrier wave is generated at or adjacent a second side of the intracorporeal tissue layer. Intracorporeal tissue layer 124 may be part of a bone wall, for example, a part of a skull or a vertebra or a long bone. Alternatively, intracorporeal tissue layer 124 may be part of a soft tissue or connective tissue, such as a blood vessel wall. Pusher head 76 may be fixated to the target portion prior to the deforming and/or may press against the target portion throughout the deforming.

Motion generator 78 is optionally driven to repeat the deforming until the distinguishable acoustic signal is generated or detected. The deforming may comprise a reciprocating movement of target portion 122 relative to the surrounding portion of tissue layer 124, such as a vibrational movement. In some embodiments, the chosen drive frequency of pusher 74 is about 1 kHz or less. Alternatively, the chosen frequency is between about 1 kHz and about 100 kHz, or between about 100 kHz and about 1 MHz, or between about 1 MHz and about 10 MHz. The acoustic signal may be analyzed to estimate at least one parameter associated with the deformation of the target portion of the bone, including any combination of frequency, echogenicity, amplitude, velocity, acceleration, temperature, elasticity and ductility.

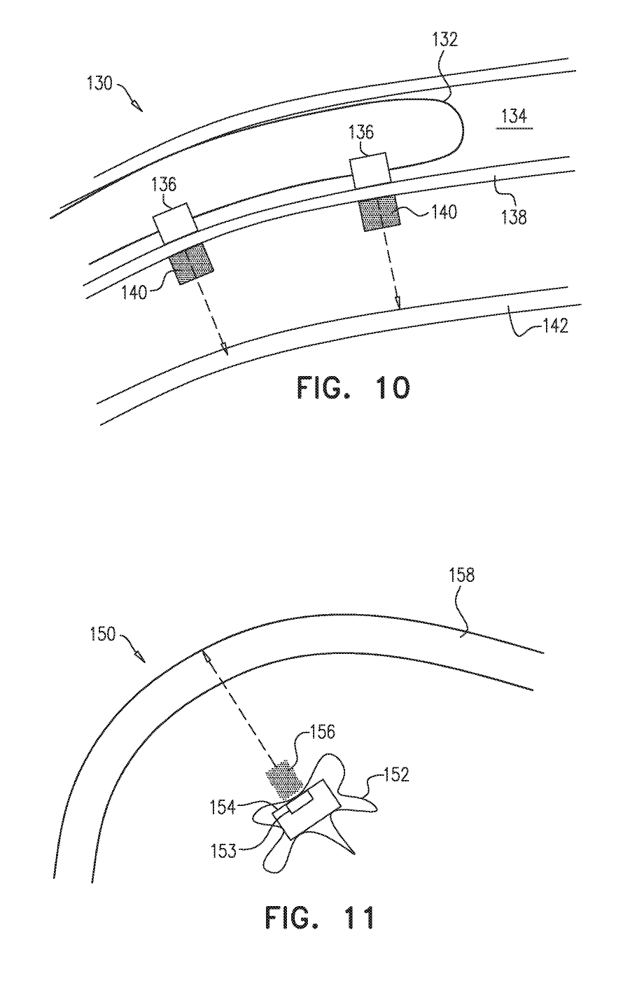

FIG. 10 is a schematic sectional view of a catheterization system 130, in accordance with another embodiment of the invention. In this embodiment, a catheter 132 is inserted into a blood vessel 134. Each of a plurality of pushers 136 mounted along a length of catheter 132 deform a small segment of wall 138 of blood vessel 134, thus generating discrete acoustical signals 140. An ultrasound probe (as shown in the preceding figures) adjacent to an outer body surface 142 detects signals 140 and thus enables accurate localization of pushers 136, thus allowing the part of catheter 132 between pushers 136 to be tracked as it progresses or is held stationary in blood vessel 134. Furthermore, when there is a substantial variance in the state or form of the visualized artifact associated with a particular pusher 136 relative to other pushers 136, conclusions can be made about the possibility of a local abnormal condition (e.g., obstruction, calcification, lesion or aneurism, for example) in proximity to the particular associated pusher 136.

FIG. 11 is a schematic sectional view of a cranial implantation system 150, in accordance with yet another embodiment of the invention. A pusher 153 is embedded in or otherwise attached to an implantable device 154, such as an implantable electrode, provided in an implantation site bounded by an intracorporeal tissue layer 152. Actuation of pusher 153 causes an acoustical signal 156 to be generated within a skull 158 of the patient. Detection of this acoustical signal from outside the skull enables implantable device 152 to be localized, optionally during delivery or after implantation. Similar techniques may be applied, for example, in surgical treatment and installation of implants in the vertebrae, as well as other bones. Means for powering and/or control can be assembled in implantation device 154, or may be activated from a different location, inside or outside the subject's body, such as by way of inductive coupling.

FIG. 12 is a schematic section illustration of a system 160 for fixating an intramedullary nail 162 in medullary cavity 28 of bone 22, in accordance with an embodiment of the invention. System 160 comprises a motion generator 166 (which may be similar in function and/or structure to motion generator 78 shown in FIG. 5 or in FIG. 6) positioned or positionable adjacent to or through a fixation opening 164 of intramedullary nail 162 within cavity 28. Motion generator 166 is configured for effecting reciprocal deformations (such as by way of vibration) of a target portion in the wall of bone 22 surrounding cavity 28 relative to the surrounding portion of the bone wall (as described with reference to target portion 122, shown in FIG. 9). In the pictured embodiment, motion generator 166 is connected to intramedullary nail 162 at or adjacent to fixation opening 164. Alternatively, however, the motion generator may be connected to an elongated member deliverable through a lumen of the intramedullary nail, as in the embodiment shown in FIG. 1.

A nail fixation template 172 is fixedly connectable at one of its ends to the proximal end of intramedullary nail 162. Template 172 incorporates at least one directional passage 174 sized and configured for aligning a nail fixator in a chosen spatial direction relative to fixation opening 164 when the template is connected as shown. Template 172 further includes means to align directional passage 174 with the chosen spatial direction, in the form of a probe holder 170 for holding and directing ultrasound probe 34, which is aligned with passage 174. Optionally, holder 170 includes the directional passage, or the ultrasound probe includes the directional passage.

In order to fixate intramedullary nail 162 in cavity 28, motion generator 166 is actuated, thus causing the target portion in the wall of bone 22 to move with a chosen magnitude and/or frequency. The target portion is deformed sufficiently in this manner, relative to surrounding portion of the bone wall, so as to generate a distinguishable acoustic signal 168 beyond the bone wall. Probe 34 detects the distinguishable acoustic signal and generates a corresponding image, as shown, for example, in FIG. 3. On this basis, the direction to the target portion relative to the extracorporeal location of probe 34 is determined either manually by the surgeon, or automatically by image processing. Directional passage 174 is then adjusted (either manually or automatically) to align in a chosen spatial direction relative to fixation opening 164.

Once this alignment is completed, a transcutaneous passage is created in soft tissue adjacent to the long bone in alignment with directional passage 174, and a hole is then drilled in the bone wall across the long bone at or adjacent to the target portion, in alignment with opening 164. A nail fixator (not shown) is delivered through the hole and fixated to intramedullary nail 162 and/or the wall of bone 22.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Surgical apparatus, comprising:
a transducer configured to be inserted into a cavity inside a bone within a body of a living subject and to engage an inner wall of the cavity at a selected location within the cavity; and
a drive circuit, which is coupled to apply a drive signal to the transducer so as to cause an echogenic movement of the bone at the selected location.

2. The apparatus according to claim 1, wherein the transducer comprises a piezoelectric crystal.

3. The apparatus according to claim 1, wherein the transducer comprises a mechanical vibrator.

4. The apparatus according to claim 1, wherein the transducer is configured to apply pulses of thermal energy to the inner wall.

5. The apparatus according to claim 1, wherein the transducer is further configured to thin the bone at the selected location.

6. The apparatus according to claim 1, and comprising an intramedullary nail, which is configured for insertion inside a medullary cavity of the bone,
wherein the transducer is mounted within the intramedullary nail in proximity to a fixation hole in the intramedullary nail, so as to engage the inner wall of the medullary cavity at the selected location in alignment with the fixation hole.

7. The apparatus according to claim 1, and comprising an elongate shaft configured for insertion into the cavity, wherein the transducer is fixed at the distal end of the shaft.

8. The apparatus according to claim 1, and comprising:
an acoustic probe, which is configured to be applied to a surface of the body in proximity to the bone, and to output a detection signal indicative of acoustical modulation due to the movement of the bone; and
a processor, which is configured to generate and output an indication of the location responsively to the detection signal.

9. The apparatus according to claim 8, wherein the acoustic probe comprises an ultrasound transducer, which is configured to direct ultrasonic waves toward the bone and to detect the acoustical modulation as a Doppler shift of the ultrasonic waves.

10. The apparatus according to claim 8, wherein the processor is configured to indicate, responsively to the detection signal, a position and direction for application of a surgical tool to the bone in order to create a hole through the bone at the location.

11. A method for localization, comprising:
bringing a transducer into engagement with a surface of a wall of a cavity inside a body of a living subject;
driving the transducer so as to cause an echogenic movement of the wall at a location of the transducer;
detecting an acoustical modulation due to the movement of the wall; and
generating and outputting an indication of the location responsively to the detected acoustical modulation.

12. The method according to claim 11, wherein the transducer comprises a piezoelectric crystal.

13. The method according to claim 11, wherein the transducer comprises a mechanical vibrator.

14. The method according to claim 11, wherein driving the transducer comprises applying pulses of thermal energy to the wall.

15. The method according to claim 11, wherein detecting the acoustical modulation comprises applying an acoustic probe to a surface of the body in proximity to the wall, and outputting from the acoustic probe a detection signal indicative of acoustical modulation due to the movement of the wall.

16. The method according to claim 15, wherein the acoustic probe comprises an ultrasound transducer, and wherein detecting the acoustical modulation comprises directing ultrasonic waves from the ultrasound transducer toward the wall, and detecting the acoustical modulation as a Doppler shift of the ultrasonic waves.

17. The method according to claim 15, wherein outputting the indication comprises indicating, responsively to the detection signal, a position and direction for application of a surgical tool to the wall in order to create a hole through the wall at the location.

18. The method according to claim 11, wherein bringing the transducer into engagement comprises fixing the transducer at the distal end of an elongate shaft, and inserting the elongate shaft into the cavity.

19. The method according to claim 11, wherein bringing the transducer into engagement comprises contacting the surface of a bone within the body, and wherein driving the transducer causes the bone to vibrate.

20-44. (canceled)

45. An implant comprising:
an implant body sized to fit in a bodily organ of a living subject surrounded by a bodily wall;
a rigid pusher with a pusher head selectively extendable from the implant body for engaging a target portion of the bodily wall; and
a motion generator operatively connected to the pusher and configured for driving the pusher head against the target portion so as to deform the target portion relative to a surrounding portion of the bodily wall sufficiently to generate a distinguishable acoustic signal.

46. (canceled)

47. The implant according to claim 45, wherein the motion generator includes at least one ultrasonic vibration actuator.

48-64. (canceled)

65. A system for fixating a long bone, the system comprising:
an intramedullary nail configured for insertion in a cavity of the long bone; and
a motion generator connectable to the intramedullary nail in a position in proximity to a fixation opening of the intramedullary nail and configured for effecting reciprocal deformations of a target portion in a bone wall surrounding the cavity relative to a surrounding portion of the bone wall.

66. The system according to claim 65, wherein the motion generator is connected to the intramedullary nail.

67. The system according to claim 65, wherein the motion generator is connected to an elongated member deliverable through a lumen of the intramedullary nail.

68-76. (canceled)
